(19) United States
(12) Patent Application Publication
(10) Pub. No.: US 2012/0296195 A1
(43) Pub. Date: Nov. 22, 2012

(54) APPARATUS AND METHOD FOR DETECTION AND MONITORING OF ELECTRONICAL ACTIVITY AND MOTION IN THE PRESENCE OF A MAGNETIC FIELD

(75) Inventors: David F. ABBOTT, Blackburn (AU); Steven W. Fleming, Greensborough (AU); Graeme D. Jackson, Balwyn (AU); Richard A.J. Masterton, Brunswick (AU)

(73) Assignee: Brain Research Institute Pty Ltd

(21) Appl. No.: 13/335,623

(22) Filed: Dec. 22, 2011

Related U.S. Application Data
(63) Continuation of application No. 12/094,126, filed on Nov. 10, 2008, now abandoned.

(30) Foreign Application Priority Data
Nov. 17, 2005 (AU) AU 2005906391

(51) Int. Cl. A61B 5/0476; A61B 5/0488; A61B 5/053; A61B 5/0402; A61B 5/055; A61B 5/04

(52) U.S. Cl. 600/411

(57) ABSTRACT

The invention generally provides a method of detecting or monitoring the characteristics (including change over time) of at least one electrical indicator of the function of a tissue in a biological organism in the presence of a magnetic field. Preferably, the magnetic field is generated by a magnetic resonance imaging (MRI) scanner. The method preferably also comprises one or more steps of detecting or monitoring sources of unwanted signal, and compensating for, or avoiding the unwanted signal. Unwanted signal includes signals arising in part due to the presence of the magnetic field, and/or directly measuring such unwanted signals for the purposes of avoidance of or compensation for the unwanted signals. Preferably, the method also enables detecting motion of the subject during the performance of the method steps discussed in the preceding two paragraphs.

This application is a Continuation of U.S. patent application Ser. No. 12/094,126, filed Nov. 10, 2008, which is a U.S. National Stage of PCT/AU06/01736, filed Nov. 17, 2006, which claims priority to Australian Patent Application 2005906391, filed Nov. 17, 2005, all of which are incorporated herein by reference in its entirety.

### FIELD OF THE INVENTION

This invention relates to apparatus and methods for the detection and monitoring of electrical activity and motion of a subject in the presence of a magnetic field. It has particular, although not exclusive application to the detection of the electrical activity of electrically excitable tissues in biological organisms, such as the bodies of mammals, and especially, such as humans. One particular use to which the invention may be applied is in monitoring and analysing brain electrical activity in humans and other mammals whilst simultaneously or concomitantly acquiring magnetic resonance images, and the background to the invention will therefore be described with particular reference to this application to which the invention is particularly suited.

### BACKGROUND TO THE INVENTION

The electrical activity of the brain and the nervous system have been studied by medical researchers for over a century. Although it was known as early as the nineteenth century that living brains have electrical activity, a German psychiatrist named Hans Berger was the first to record this activity in humans, in the late 1920s.

The development of the electroencephalogram (EEG) was a significant development in the study of brain function. The EEG is a record of changes in the electrical potential difference of the brain of a subject between two points on the scalp. The EEG is taken non-invasively, and allows an observer to follow electrical impulses across the surface of the brain and to observe changes over time. As a general rule, an EEG can provide an indication of the subject's state of consciousness—namely, whether the subject is asleep, awake or anaesthetised—because the characteristic patterns of voltage differ for each of these states. A major drawback of the EEG however, is that the technique cannot show the structures or the anatomy of the brain. Nor can the EEG indicate which specific regions of the brain perform particular functions.

More recently, other non-invasive techniques have been developed by medical researchers, for studying and monitoring brain function. One of the techniques that has gained wide acceptance in the last three decades is the use of magnetic resonance imaging (MRI). MRI is based on the principles of nuclear magnetic resonance (NMR), a spectroscopic technique used by scientists to obtain microscopic chemical and physical information about molecules. Magnetic resonance imaging is based on the absorption and emission of energy in the radio frequency range of the electromagnetic spectrum.

Cognitive neuroscience was revolutionised in the early 1990s by the introduction of the blood oxygenation level dependent (‘BOLD’) method for identifying active neural regions through changes to the activity caused in local deoxyhaemoglobin concentration (Ogawa, Lee et al. 1990; Kwong, Belliveau et al. 1992; Ogawa, Lee et al. 2000). Implementation of this method came to be known as functional Magnetic Resonance Imaging (‘fMRI’). This technique allows mapping of the function of the various regions of the brain. fMRI is used to visualise brain function, by monitoring and recording changes in visual images that in turn, correspond to changes in the chemical composition of, or the flow of fluids in areas of the brain or other tissues that occur over relatively short time spans (typically, time spans of seconds to minutes). For example, in the brain, blood perfusion is thought to be related to neuronal activity, and accordingly, fMRI can be used to examine the activity of the brain when a subject performs a specific task or is subjected to specific stimuli. The fundamental principle of fMRI is to take a series of images of the organ or tissue under study (often, but not always the brain) in rapid succession and to analyse the images for differences among them.

Nowadays, clinicians generally understand the physiology of the areas of the brain where functions such as speech, sensation and memory, are controlled. The precise locations vary however, from individual to individual. Injuries or diseases to the central nervous system can even cause control of certain functions to shift to parts of the brain where they would not normally be controlled in a normal subject. fMRI can therefore be used as a tool by clinicians to examine the anatomy, physiology and pathology of the brain, and can also help them to identify with reasonable precision, which part(s) of the brain are handling functions such as thought, speech, movement, or sensation. Information about matters such as these can be critically important in assisting a clinician to plan for surgery, radiotherapy, or other treatment regimens for a particular patient.

The advent of fMRI has considerably expanded the clinician's palette of tools for understanding the function of organs such as the brain. In some instances however, fMRI alone will not provide a sufficient diagnostic tool, and additional tools for understanding brain function need to be used. For example, in clinical practice, there are not infrequently situations where, in addition to capturing images of the brain via fMRI, other indicators of brain function need to be measured by using the EEG. A typical example is in the diagnosis and/or treatment of epilepsy, where capturing EEG data (and particularly data concerning the activity of epileptic foci in the patient's brain) can be very useful (and often, critical) to the clinician. It is often generally inconvenient to require a patient to undergo separate fMRI imaging and EEG procedures, as doing so can compound the feeling of unease that many patients experience about undergoing medical diagnostic procedures. Moreover, the simultaneous/concomitant capture of fMRI and EEG data can yield useful information for the clinician, to assist in diagnosing the patient's condition. Capturing that information often also assists in (and in some instances, is critical to) developing suitable treatment regimens. There are some difficulties associated with simultaneously capturing fMRI and EEG data however. Those difficulties include the following.

One of the main difficulties is that artifacts can arise in the process of acquiring the EEG signal. An artifact is generally any feature which appears in the results displayed from the EEG acquisition process, but which does not represent the EEG signal derived from the patient's brain. Accordingly, artifacts have the potential to distort or even obliterate the true EEG signal, thus rendering the process of capturing EEG data from the patient either inaccurate, or at worst, useless to the clinician.

Artifacts may arise from a number of sources. In general, the most significant artifact encountered when collecting EEG data in a patient, is the gradient induced artifact. This artifact is generated by the transient magnetic field applied within the MRI scanner during an MRI or fMRI imaging procedure. The gradient induced artifact is caused by exposure of conductive loops formed by the EEG leads and scalp to the changing magnetic field.

Other artifacts arise due to the large static magnetic field that is always extant within an MRI scanner. Any movement of a loop of conductor material in a static magnetic field generally will induce a voltage. Thus, where an EEG recording is sought to be made in the course of capturing fMRI data, artifacts can be induced by (for example), movements of the patient's head (and corresponding movement in the attached EEG leads) in the MRI scanner chamber, or by vibration of the EEG leads due to scanner noise. Motion of the subject also has a deleterious effect on the MRI acquisition.

The voltage artifacts induced in the EEG leads by sources such as those mentioned in the preceding paragraphs are typically many orders of magnitude larger than the EEG signal. They are usually the most difficult source of artifact noise that must be addressed. In addition, artifact noise can result from the EEG amplifiers that typically must be used, or from other equipment associated with the data acquisition system. Further, care must be taken to avoid inflicting burns to the patient, as the radio frequency (RF) pulse generated by the MRI equipment during scanning will induce current in low impedance loops. The larger the loop, the larger the voltage induced.

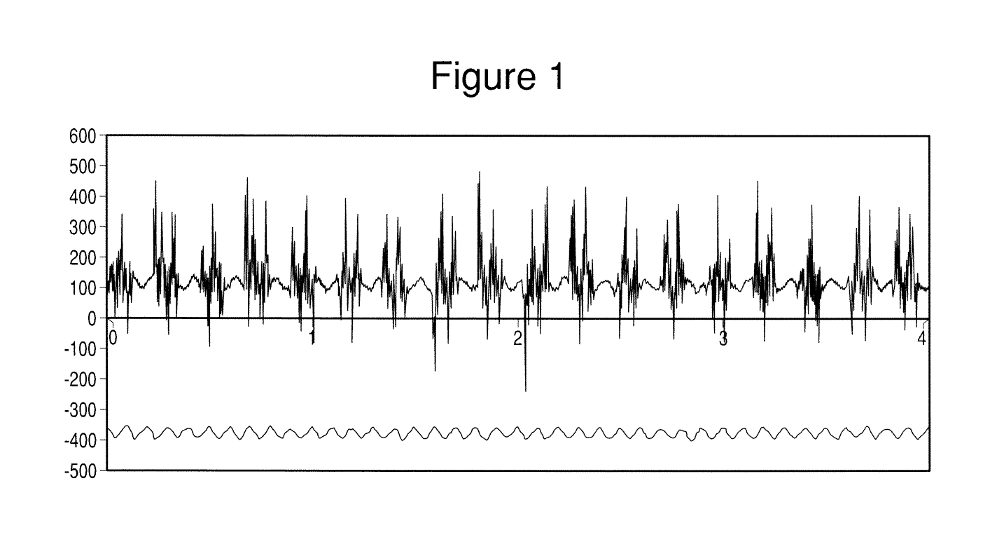

The strength of the static (i.e. constant and always-on) magnetic field within the chamber of most MRI scanners is in the range of between 0.5 tesla and 7.0 tesla, although higher and lower fields are possible. Typically, the static magnetic field within the chamber of most modern MRI scanners in clinical use is of the order of 1.5 to 3.0 tesla. Transient magnetic field gradients used to generate the imaging data can typically approach 70 mT/m, although gradient strengths more than twice that are available in some systems. The magnetic gradients available in typical MRI systems are sufficient to generate an artifact which has an amplitude such as to obscure the EEG signal completely, whose voltage amplitude—by comparison—is relatively small. The EEG signal thus invariably requires amplification. The signal also has high source impedance due to various biological factors, including the impedance from the scalp to the EEG lead. The relative voltage amplitude of the artifact compared to the EEG signal is illustrated in FIG. 1. As will be seen, without the use of counter-measures, the gradient induced artifact would typically obscure the EEG signal altogether, thus rendering the capture of EEG data from the patient completely futile. Generally less intense than the gradient artefact is the artefact that arises from motion of loops of conducting material in the static magnetic field. These artifacts however can cause unpredictable signal on the EEG that in some cases is recognisable as false signal that can obscure the true EEG, and in other cases may appear focally, bilaterally or unilaterally and can sometimes be confused for epileptiform activity.

One way in which the gradient artifact problem might be addressed would be by seeking to amplify the ‘true’ EEG signal, and/or to use techniques that seek to subtract the artifact from the true EEG signal. In practice however, such techniques can be difficult to implement, as (amongst other things) the mathematical calculations required in order to generate an EEG tracing which subtracts the artifact noise are often complex, and rely on assumptions about the physical parameters that applied during the scan, which may not always hold true. To illustrate the difficulties associated with these techniques, converting an analog EEG signal to a digital one continuously in the course of the scan requires that signal artifact generated during gradient transitions be discarded, which can be difficult. This requires amplification equipment that does not saturate during scanning, and which has sufficient frequency response to follow the gradient artifact, as well as sufficient temporal resolution and dynamic range to measure the EEG signal and the artifact

Accordingly, the resultant tracing cannot always be relied upon as providing an accurate representation of the true EEG activity in the patient. In addition, the equipment that must be used in order to carry out these techniques can be expensive, and thus not as readily available for widespread clinical use as would otherwise be desirable. Further, equipment that is used in order to carry out these techniques may require the use of non-conventional EEG equipment, which is generally not preferred.

Another way in which the gradient artifact problem might be addressed may be by using a specially designed MRI acquisition sequence in which there are regular periods of absence of applied magnetic field gradients. Synchronising discrete periodic sampling of the EEG to coincide with the periods of absence of magnetic field gradients may then serve to avoid the gradient-induced artifact. Unfortunately this may require that non-standard MRI sequences are employed that have sufficient known non-gradient active periods to allow sampling of the EEG during the gradient-off periods. Further, the use of non-conventional EEG equipment may also be required, for unless high-frequency and high-bandwidth amplifiers are used as described above, the recovery-time after saturation of standard EEG amplifiers by the gradient-induced signal may compromise the accuracy of measurements made during the gradient-off periods. Thus this method may suffer many of the limitations of the subtraction method described in the preceding paragraph.

The present invention therefore aims to provide methods and apparatus for detecting and monitoring the electrical activity of electrically excitable tissues (such as the brain) of a subject, and thereby, to address one or more of the prior art problems previously discussed. In particular, there is a clinical and research need to capture EEG and MRI data simultaneously, and the invention specifically seeks to address this need.

### GENERAL DISCLOSURE OF THE INVENTION

The invention generally provides a method of detecting or monitoring the characteristics (including change over time) of at least one electrical indicator of the function of a tissue in a biological organism in the presence of a magnetic field.

Preferably, the magnetic field is generated by a magnetic resonance imaging (MRI) scanner. The method preferably also comprises one or more steps of detecting or monitoring sources of unwanted signal, and compensating for, or avoiding the unwanted signal. Unwanted signal includes signals arising in part due to the presence of the magnetic field, and/or directly measuring such unwanted signals for the purposes of avoidance of or compensation for the unwanted signals.

Preferably, the method also enables detecting motion of the subject during the performance of the method steps discussed in the preceding two paragraphs.

The biological organism is preferably a mammal. The mammal may be a human or a non-human subject.

The tissue is preferably an electrically excitable tissue. The tissue may be a neural tissue. Preferably, the neural tissue is the central nervous system of the subject, or a part of the central nervous system. In a particularly preferred embodiment of the invention, the tissue is the brain of the subject, or a part of the brain.

Alternatively, the tissue may be a peripheral tissue, such as a peripheral nerve or part of a peripheral nerve.

Alternatively again, the tissue may be another body tissue or organ which conducts electrical activity. In this further embodiment of the invention the tissue may be, for example, the skin of the subject. Alternatively the tissue may be the heart or a part of the heart of the subject. For example, the tissue might be the Purkinje system of the subject's heart, or a part of that system. Alternatively, the tissue could be cardiac muscle tissue in the heart of the patient.

Alternatively again, the tissue could be a muscle tissue. In this case, the tissue could be either a skeletal muscle tissue, a smooth muscle tissue or, as mentioned in the preceding paragraph, a cardiac muscle tissue.

The magnetic field in which the biological organism is placed could be a static and/or a time varying magnetic field. The magnetic field may be generated by an MRI scanner or other instrument. By way of an example, in the case of an MRI scanner the magnetic field may comprise both a static and time-varying component, the static component being always present and the time-varying component only being present during image acquisition by the MRI scanner.

In a particular aspect of the invention, the MRI scanner itself may measure the electrical activity of an electrically excitable tissue. In this aspect, preferably the MRI sequence may be a spin-echo sequence or a gradient-echo acquisition sequence.

In all preferred applications of the invention, an MRI scanner comprises, or co-operates with means suitable for detecting the electrical activity in the specific tissue under study. For example, in a subject undergoing a brain scan, the subject could be fitted with one or more electrodes or like means for detecting brain electrical or other physiological activity.

Preferably, the means by which changes in the electrical indicator are detected comprise the use of electrodes attached or located near to the biological organism. For example, an electroencephalogram and/or other observations (for example, readings on the subject's blood pressure or blood chemistry) on the subject could be taken.

Alternatively, the MRI scanner itself may be used to directly detect electrical activity of interest. In this case, the MRI measure either may, or may not, also be combined with the use of electrodes or other means as described above.

The electrical indicator may be either (or both):
(a) a direct electrical indicator of characteristics of function of the tissue; or
(b) an indirect electrical indicator of characteristics of function of the tissue.
By way of example, preferred direct electrical indicators of tissue function include inherent indicators of electrical function of the tissue, such as:
   the Electroencephalogram (EEG);
   the Electrocardiogram (ECG);
   the Electromyogram (MCG); and
   measurements of skin or other tissue conductance.

In contrast, indirect electrical indicators include measurements of other characteristics of tissue function, such as (for example), temperature, tissue oxygenation levels, tissue or body fluid chemistry, where the measurement of the characteristic is made by means which covert the measurement to an electrical signal which can be detected and/or monitored in accordance with the apparatus and method aspects of the invention.

Preferably, the method would permit the simultaneous or concomitant detection and recording of changes over time in the MRI acquisition sequence of the tissue under observation, as well as changes over time in the electrical observations on the subject.

Preferably, the output of readings taken by the MRI scanner and the other detection means would be displayed on a display means, such as a computer monitor or other visual monitor. The output would preferably also be recorded via recording means. Such means could take the form of an electronic file stored on a computer hard disk or another electronic recording medium (such as a compact disc, Digital Versatile Disk (DVD), or other means capable of being played back as and when desired). Alternatively, the output of the readings could be displayed via a printing means, such as a computer printer.

In a preferred embodiment of the invention, the method comprises:
(i) detecting or monitoring changes in the patient's brain or central nervous system by the use of MRI techniques; and
(ii) simultaneously or concomitantly, recording the EEG from the patient.

In a particularly preferred version of this embodiment of the invention, the method comprises the simultaneous or concomitant capture of functional MRI (fMRI) and EEG data from the patient. In this version, the method comprises detecting or monitoring changes in the characteristics of at least one electrical indicator of the function of the tissue (typically, the brain or central nervous system) by selectively sampling readings taken from the tissue over a period of time.

Preferably, the method comprises the step of:
(i) capturing MRI (and particularly fMRI) readings taken from the patient; and
(ii) selectively sampling readings taken from an EEG taken simultaneously or concomitantly with the MRI or fMRI scan.

In a particularly preferred embodiment of the invention, the step of selectively sampling the EEG readings taken from the patient comprises the use of means which are able to distinguish between:
(a) the true EEG signal; and
(b) artifacts or signals which do not represent the EEG signal.

In a yet further preferred embodiment of the invention, those means capture sample readings from the EEG in the patient, during periods of time when artifacts in the readings are either absent, or are substantially absent.

Preferably further, the method of the invention may also be performed in association with:
filtering means (to filter unwanted data); and/or
post-data capture processing means,

in order to present data captured from the patient that represent the most accurate possible readings taken from the EEG and the MRI.

Preferably further, recording means in addition to the EEG may be used to capture sample readings that contain a signal that is absent of true EEG signal but which contains motion-related signal and/or other artifact similar to that contained in the EEG recording. The artifact signal directly recorded by this recording means may be used to advantage in the filtering and/or post-data capture processing means described above.

In yet further applications, it might also be useful for the method to be performed in association with one or more other procedures being carried out on the subject. For example, it would be beneficial in some instances, if the method aspect of the invention were performed in conjunction with a surgical or other therapeutic procedure being performed on the subject.

In further embodiments of the invention, the method aspect of the invention could be used to detect the impact of specific stimuli on changes in the MRI signal and/or electrical signals (comprising measurements of either or both direct and indirect electrical indicators) in the tissue under study in the subject. Such stimuli could take the form of:
Psychological stimuli, such as stimuli designed to induce a specific psychological or emotional state in the subject;
Physiological stimuli, such as visual, aural, olfactory, proprioceptive, nociceptive, or temperature-related stimuli (eg, the application of heat or cold temperatures to the subject); or
Pharmacological stimuli, such as those produced by applying one or more pharmacological agents to the subject.

The invention also provides apparatus for detecting or monitoring the electrical activity of an electrically excitable tissue in a biological organism, the apparatus comprising means for detecting or monitoring in the tissue, changes in the characteristics of at least one electrical indicator of the function of that tissue, over a period of time.

Preferably, the apparatus comprises means which permit the capture of at least one electrical indicator of the function of the tissue, and whereby those means are able to co-operate with apparatus used for carrying out other procedures in the patient.

Preferably, the apparatus comprises means for detecting the EEG in the patient, while the patient undergoes an MRI (and particularly an fMRI procedure).

In a particularly preferred embodiment, the apparatus comprises a skull-cap fitted with electrodes or like apparatus for detecting brain electrical activity, with the electrodes interfaced to a ‘head-box’ containing electronic circuits which selectively sample and filter the EEG signal, providing an output that can be recorded by conventional EEG recording equipment with little, if any, modification. The electrodes and head-box, or like apparatus, would be particularly configured so as to be capable of being used inside an MRI scanner, during an MRI scanning procedure.

An aspect of the invention provides means for selectively sampling from the EEG readings, during the course of a simultaneous/concomitant EEG/MRI scanning procedure performed on the patient. Preferably, those means comprise means which are able to distinguish between:
(a) the true EEG signal captured from the patient; and
(b) artifacts or signals which do not represent the EEG signal, for example those signals that may be induced by a changing magnetic field induced by the MRI scanner.

Preferably further, those means are able to capture readings from the patient's EEG during periods of time when artifacts in the EEG readings are either absent or are substantially absent.

A further aspect of the invention provides means for recording and correcting for cardio-ballistic and motion-related artifactual signal induced in the electrical recording. Preferably these means comprise an additional recording means that may be used to capture sample electrical readings that contain a signal that is absent of true signal of interest but which contains artifact similar to that contaminating the electrical recording. The artifact signal directly recorded by this additional recording means is preferably used by a filtering and/or post-data capture processing means to substantially reduce the effect of the artifact in the electrical recording.

A further aspect of the invention provides for the use of the artifact signal, directly recorded by the additional recording means described in the preceding paragraph, in MRI filtering and/or post-data capture processing means to substantially reduce the effect of motion artifact in the MRI recording.

In an especially preferred embodiment, the invention provides an integrated system for simultaneously/concomitantly recording MRI and EEG data, and which includes one or more of the apparatus and method features referred to in the preceding paragraphs.

### DETAILED DESCRIPTION OF A PREFERRED EMBODIMENT OF THE INVENTION

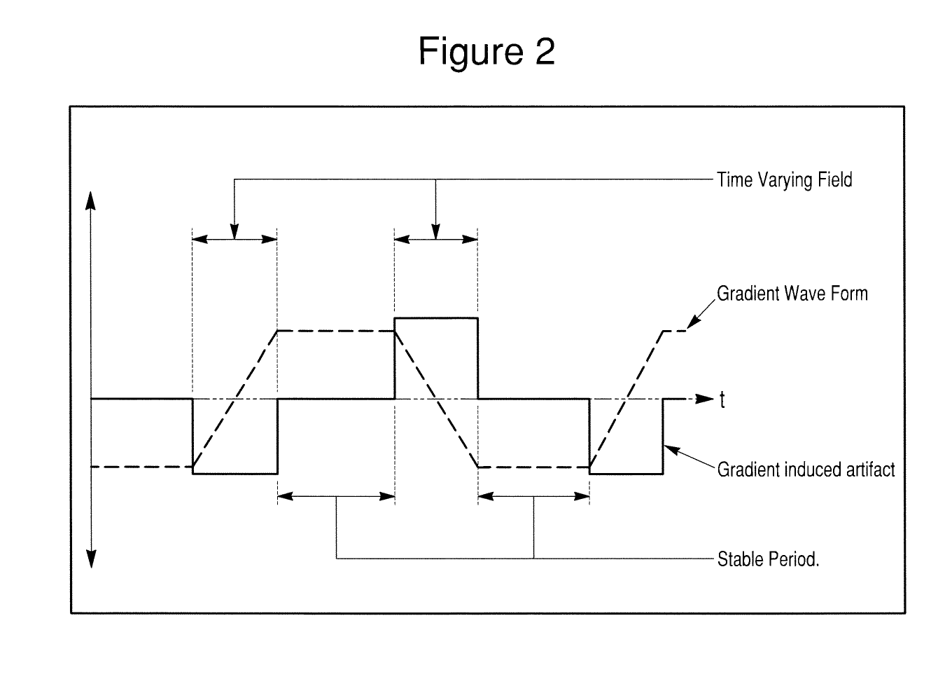

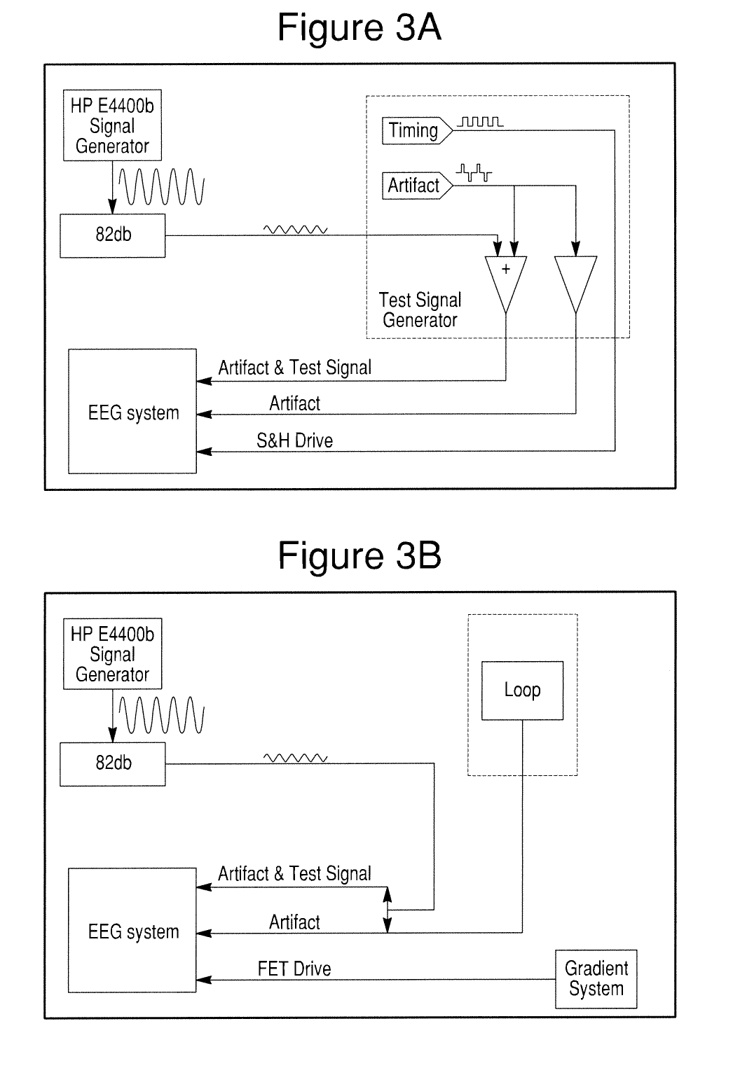

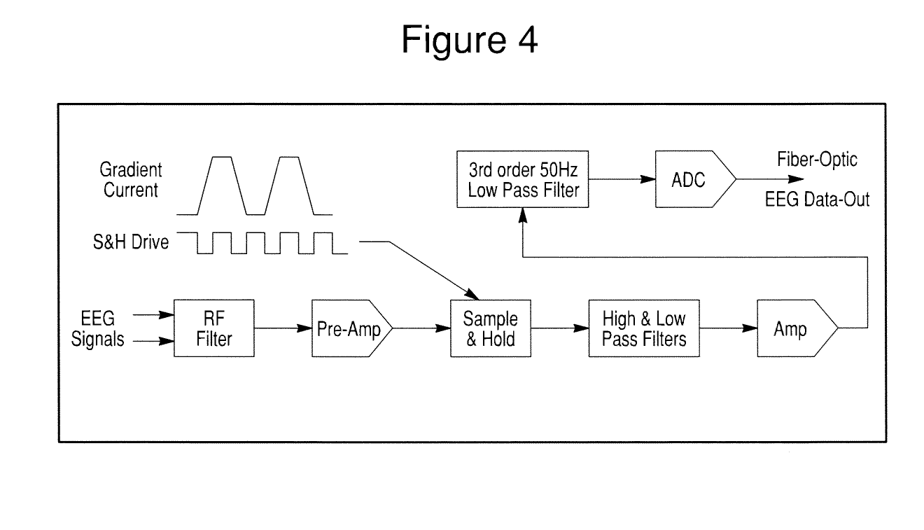

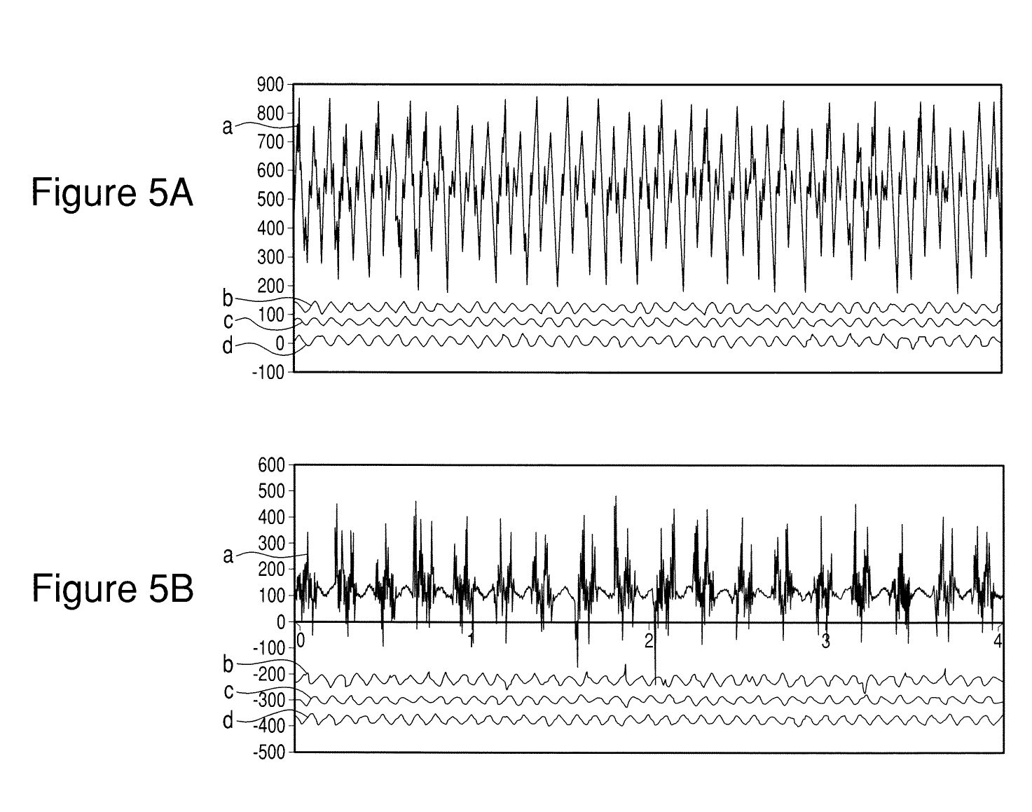

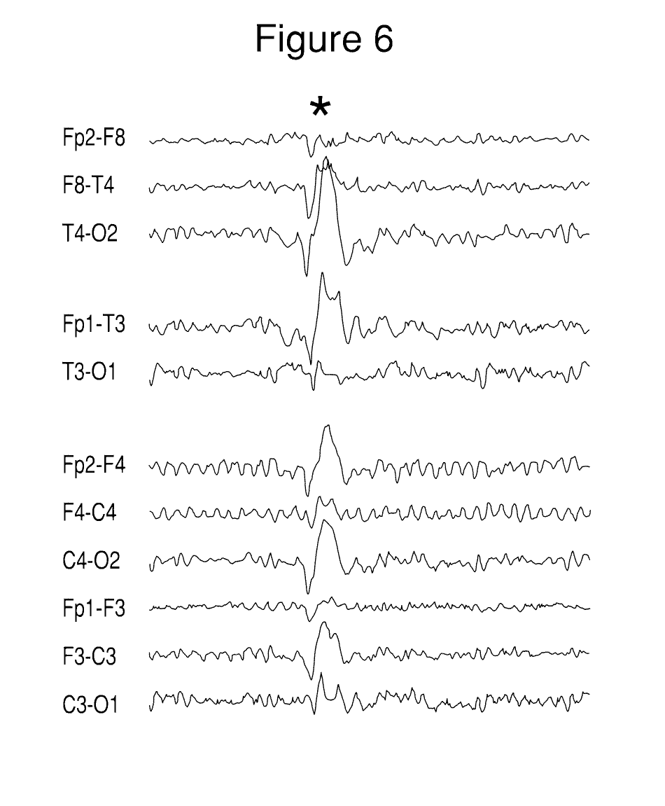

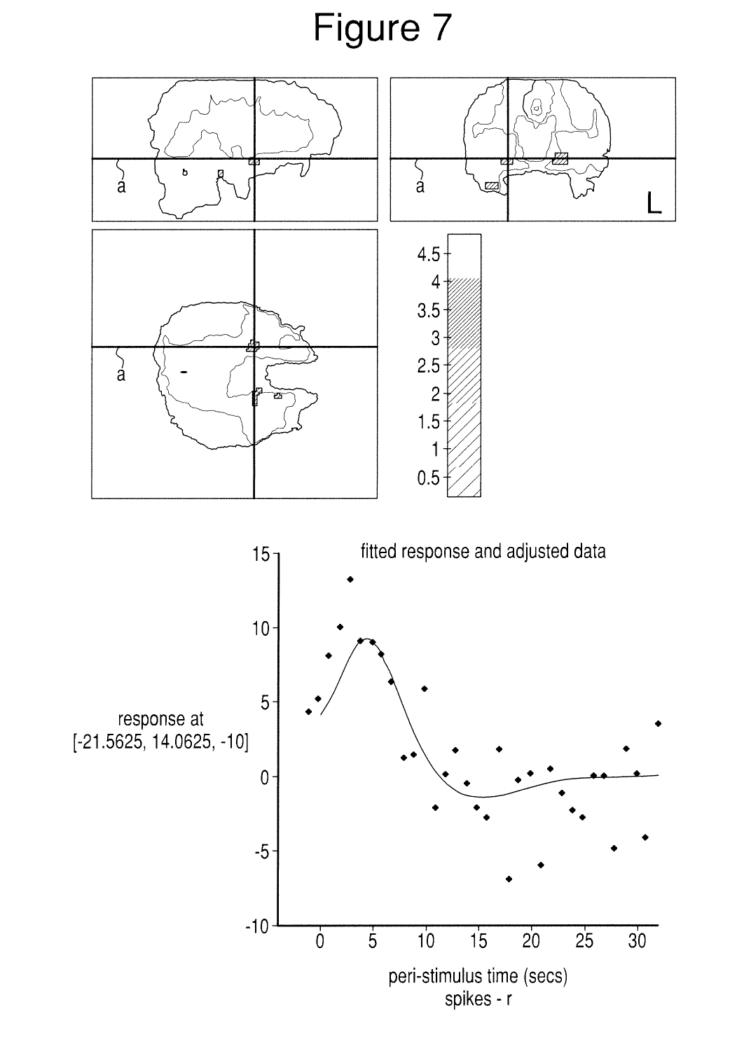

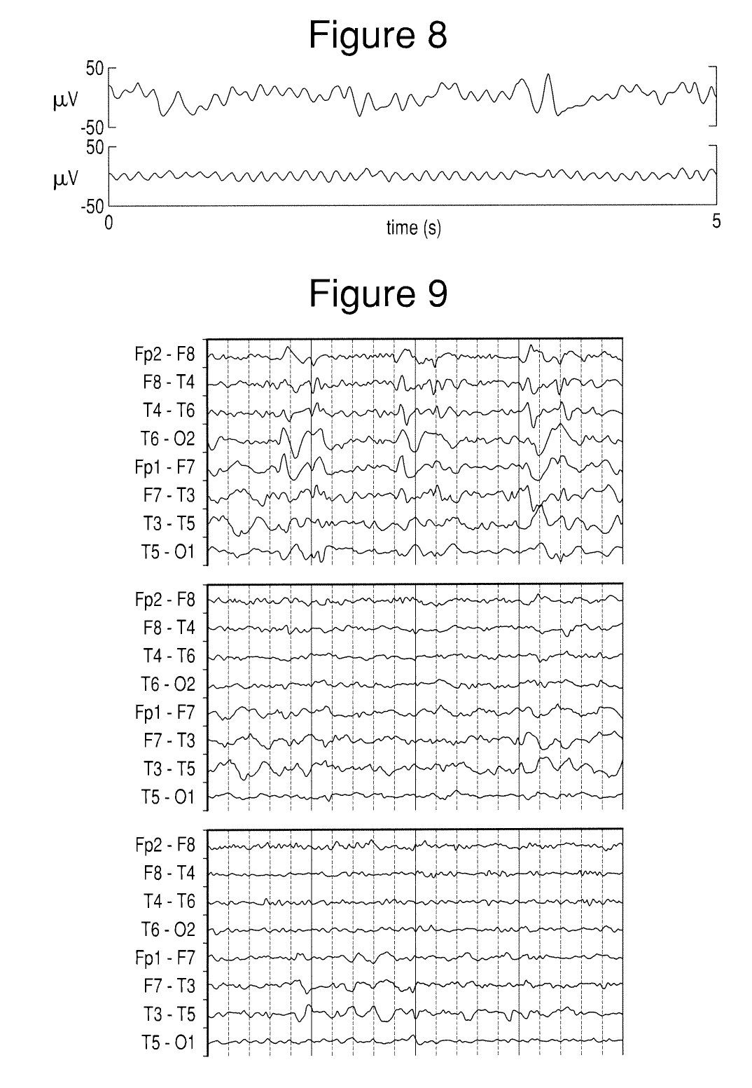

A preferred embodiment of the invention will now be described by way of example only with reference to the accompanying drawings, in which:

| | |
|---|---|
| FIG. 1 | represents the relative voltage amplitudes of the EEG and the |
| | gradient artifact typically encountered during MRI scanning; |
| FIG. 2 | represents a diagram which schematically depicts some |
| | examples of periods of time during a combined EEG -MRI |
| | scan when capture of true EEG data from a patient, in |
| | accordance with the invention, would be possible; |
| FIG. 3 | represents a circuit diagram for a signal generator for use in |
| | accordance with the invention; |
| FIG. 4 | represents a schematic diagram of the attributes of the \grave{ }‘head |
| | box’ apparatus aspect of the invention; |
| FIG. 5 | represents a set of readings taken from a test version of the |
| | combined EEG - fMRI system constructed in accordance with |
| | the invention; |
| FIG. 6 | represents the results from readings taken in a combined |
| | EEG - fMRI procedure (in accordance with the invention |
| | performed on a 9 year old boy with epilepsy); and |
| FIG. 7 | depicts an fMRI brain slice image, depicting epileptic focal |
| | activity in the patient whose EEG readings are shown in FIG. |
| | 6. |
| FIG. 8 | depicts an actual recording of a simulated EEG signal |
| | (sinusoid test signal), taken from a subject moving inside an |
| | MRI scanner. Nodding produced a large artefact (top). After |
| | application of the applicant's real-time post-processing |
| | method to reduce cardio-ballistic and motion-related artifact, |
| | the artefact was significantly reduced whilst preserving the |
| | sinusoid test signal (bottom). |
| FIG. 9 | Bottom image depicts an EEG trace of another patient |
| | recorded outside of the MRI scanner, depicting focal inter- |
| | ictal activity (i.e free from magnet-related cardio-ballistic |
| | artifact); top image depicts an EEG recording from the same |
| | subject whilst inside the MRI scanner with the head-box |
| | aspect of the invention enabled; middle image depicts the |
| | same within-MRI EEG recording after correction for the |
| | measured cardio-ballistic and motion artifact. The artifact |
| | visible in the top image is virtually eliminated from the |
| | resultant EEG, and importantly the true inter-ical spiking |
| | activity of the epileptic patient is preserved. |

Referring now to the drawings, FIG. 1 depicts the relative magnitude of the voltage amplitudes of the EEG and the gradient artifact (respectively) that is typically encountered during MRI scanning. As will be seen, the typical amplitude of the gradient artifact is many magnitudes greater than the amplitude of the EEG voltage. Accordingly, and as described earlier, this means that during the conduct of an MRI scan, taking EEG readings is difficult for this reason alone.

The applicant's approach to recording of electrical signals via electrodes takes advantage of the relative stability of the magnetic field between periods of gradient transitions. The applicant's approach therefore ignores the signal during periods of gradient induced artifact. The applicant's approach to solving the difficulty of capturing EEG data during MRI scanning is summarised in FIG. 2, where the effect of the gradient induced artifact on measurement of the EEG is schematically demonstrated. As depicted in FIG. 2, during the period of a scan, the gradient induced artifact results in the voltage amplitude remaining relatively stable during certain periods (the periods concerned being designated ‘A’ in FIG. 2), and at other times, the gradient is unstable (the unstable periods are depicted in FIG. 2, generally by reference to the letter ‘B’). During the time periods denoted in ‘A’ in FIG. 1, relatively accurate recording of the EEG is possible. Accordingly, the applicant has devised a method (and apparatus for use in the method) whereby EEG measurements can be taken during those periods when the gradient is relatively stable.

The applicant has tested the theoretical possibility of recording EEG data during periods of relative stability in:
(a) test electronic environments;
(b) in an animal model (sheep); and
(c) in at least one human patient (the results reported here are those taken on a nine year old male suffering form epilepsy).

The applicant's approach to recording and filtering of cardio-ballistic and motion-related artifact uses one or more electrodes that do not make electrical contact with the subject. These additional electrodes record signal that is substantially free of signal of interest, but which remains contaminated with artifacts similar to that contaminating the recordings of the signal of interest. Combinations of the measurements taken from the additional electrodes are then fitted to the recorded measurements of the signal of interest, and the fitted artifact is then subtracted from the recorded measurements of the signal of interest.

The methods and equipment used, and the results obtained are discussed in the following description.

Equipment

All the tests reported in this document were performed on a General Electric Medical Systems (Milwaukee, Wis.) 3 tesla VH/I system. The system was equipped with 150T/M/S 40 mT gradients, and a 3T940 General Electric Magnex magnet. EEG data for the applicant's first implementation were collected on a Compumedics e Series 2 channel system. A second implementation of the invention used EEG recording hardware and software designed and built by the applicants.

Measurements were taken on a Tektronix Inc (Beaverton, Oreg.) TDS032 300 MHz digital oscilloscope. A Hewlett Packard E4400b signal generator was used to generate sinusoidal test signals.

The applicant used silver/silver chloride coated plastic electrodes (Meditec, S, Polo dl Torrile (PR), Italy) connected to carbon fibre leads constructed by the applicant.

With these leads and electrodes, the applicant was able to achieve less than 5K Ohms impedance per electrode and minimal artifacts and no subject discomfort from heating. The applicant further used high resistance carbon fibre leads to limit current flow, and to run the cables from the head out the back of the magnet to avoid loops with the patient, and use high impedance radio frequency filters.

EEG signals were recorded referenced to a central electrode (Pz) using a head-box constructed by the applicant which samples the EEG at 256 Hz, digitises it into 12-bits and transmits over a fibre-optic cable to a recording system outside the scanner room. The head box provided the hardware filtering of artifacts associated with fMRI image acquisition as described above and in FIG. 4.

In a second implementation of the invention, four additional carbon fibre loops with an approximate diameter of 10 cm each and with a common ground were sewn into place upon the surface of a neoprene EEG cap that held the EEG electrodes in place. Three of the additional loops were roughly oriented in the axial, sagittal and coronal planes respectively and the fourth loop was oriented obliquely to the other three. The loops are subject to induced voltages from movement when in the magnetic field, but as they are electrically isolated form the scalp they do not record the true EEG signal of interest. The oblique loop was used for the purposes of simulation experiments conducted by the applicants and is not actually used in the filtering process.

The additional leads were connected to the head box in the same manner as the EEG electrode leads. In this particular implementation of the invention the EEG and motion signals output by the head-box were recorded by hardware designed and built by the applicants (although standard EEG acquisition hardware would suffice), and the recorded signal was input directly into EEG display software written by the applicants for use by the applicant's implementation of a real-time cardio-ballistic and motion filter.

The real-time filter implemented by the applicants uses a Multi-channel Recursive Least Squares (M-RLS) algorithm based upon the method described in [Bouchard, M. and S. Quednau, Multichannel recursive-least-squares algorithms and fast-transversal-filter algorithms for active noise control and sound reproduction systems. 2000. 8(5): p. 606]. The applicant's implementation uses the signals from three motion sensors to form an estimate of the cardio-ballistic and motion-related artifact and then subtracts this from the recorded electrical signal of interest.

Testing the System

Initial testing of the gradient artifact removal method was performed with a test signal generator. This avoided cardio-ballistic and movement artifacts. The applicant designed a test signal generator (depicted schematically in FIG. 3) to produce a wave form similar to the readout gradient artifact, and a wave form that emulated alpha waves as might be measured from the brain of a human patient. The alpha wave was selected because it is simple to generate with electronics and for final testing in a volunteer (whose eyes would be closed). This allowed the applicant to undertake most of the development of the system in the laboratory. The testing was performed in four stages, namely:
1 where the system was connected to the test signal generator, with an emulated alpha test signal;
2. where the system was connected to a test loop in the MRI with an emulated alpha test signal (which allowed the applicant to test the system with real gradient induced artifacts, but with no movement or cardio-ballistic artifacts);
3. with the system connected to a sheep with intra-cranial electrodes (which allowed the applicant to test the system with a large EEG signal with real spikes and real gradient artifact); and
4. where the system was connected to a volunteer.

Initial testing of the cardio-ballistic and motion artifact removal method was performed by imaging a healthy volunteer. The volunteer was fitted with the EEG cap and attached motion-recording loops and placed in the MR scanner. A sinusoidal signal generator was placed in series with the oblique motion loop lead between the volunteer and the head box. The signal generator produced a 10 Hz sinusoid with 20 μV peak-to-peak amplitude—providing a crude simulation of the human alpha rhythm. The subject was instructed to lie still for a period and then separately nod (back and forth movement), sway (side to side movement) and twist (rotating neck) their head slightly for short periods of approximately one minute duration.

fMRI Parameters

In subjects, fMRI was performed using a whole brain (25 slice) gradient-recalled echo-planar imaging technique, the parameters for which were:

TE=40 m SEC

TR=300 m SEC

Acquisition sequence=128 by 128

Flip angle=60°

4 mm thick slices.

Results

FIG. 5 depicts test data recorded on the applicant's EEG system. The top trace shows actual gradient artifact and 40 microvolt, 10 hertz sinusoidal test signal. The next trace is the same signal with correction switched on. The next is with a 15 hertz filter applied, and the final (almost identical) trace is the signal, when no artifact is switched on.

The applicant recently investigated a nine year old boy, with a two year history of epilepsy. His conventional structural MRI scan showed no lesion. However, ictal EEG and seizure semiology pointed to a seizure focus in the right amygdala. At 3 tesla, an MRI/EEG study was performed, but due to the patient's young age, only 20 minutes of continuous fMRI recording was possible. FIG. 6 depicts the EEG recorded during scanning, with a clearly visible spike. The top panel depicts the patient's epileptic discharges outside the MRI scanner (left side) and during fMRI scanning (right side). Three typical spikes were captured. Event related functional image analysis disclosed significant activation associated spikes located in the right amygdala. This is depicted in FIG. 7, where the analysis showed activation in the right amygdala, and less pronounced in the left amygdala. The site of the seizure focus was consistent with the electro clinical features observed, and epilepsy surgery with removal of the right amygdala was suggested.

FIG. 8 depicts test data recoded on a second version of the applicant's EEG system. In addition to improvements in the applicant's implementation of the head-box, this version of the system included the applicant's implementation of the cardioballistic artifact measurement and correction method. The top image of FIG. 8 shows an EEG trace of a healthy human subject whilst the subject was moving his/her head in the MRI scanner and whilst a 10 Hz sinusoidal test signal was placed in series with the test loop. Movement related artifact is prominent, as is cardio-ballistic artifact. The bottom image of FIG. 8 shows the EEG trace after correction for the measured motion-related and cardio-ballistic artifact, showing a trace virtually free of artifact and in which the test-signal is preserved. FIG. 9 shows an EEG trace of yet another subject; this subject having epilepsy with clearly visible inter-ictal spikes on the EEG. The bottom image of FIG. 9 shows the EEG trace recorded from the subject outside the MRI (i.e free from magnet-related cardio-ballistic artifact); the top image shows an EEG recording from the same subject inside the MRI with the head-box aspect of the invention enabled but without the cardio-ballistic correction enabled; the middle image of FIG. 9 shows the same EEG recording after correction for the measured cardio-ballistic artifact. The artifacts visible in the top image are virtually eliminated from the resultant EEG, but importantly the true inter-ical spiking activity of the epileptic patient (as evident in the bottom image) is preserved in the middle image by the applicant's filtering method and their implementation thereof.

It is to be understood that wherever used in this specification (including both the description and the claims), forms of the word ‘comprise’ are equivalent in meaning to the corresponding forms of the word ‘include’, and are thus not to be taken as excluding or implying the exclusion of a feature or integer.

It will be also understood that the invention disclosed in this specification extends to all combinations of two or more of the individual features mentioned or evident from or implicit in the text of this specification or the accompanying drawings. All such different combinations constitute various alternative aspects of the invention.

The claims defining the invention are as follows:

1. A method of detecting or monitoring the characteristics of at least one electrical indicator of the function of a tissue in a biological organism in the presence of a magnetic field over time.

2. A method as claimed in claim 1, in which the magnetic field is generated by a magnetic resonance imaging scanner.

3. A method as claimed in claim 1 in which the method additionally comprises one or more of the steps of:
(a) detecting or monitoring sources of unwanted signal or signal noise; and
(b) compensating for, or avoiding the unwanted signal or signal noise.

4. A method as claimed in claim 3 in which the unwanted signal or signal noise comprises signals associated with the presence of the magnetic field.

5. A method as claimed in claim 3 in which the unwanted signal or signal noise is avoided or eliminated by mathematical computation.

6. A method as claimed in claim 1, in which, in the performance of the method in motion of the biological organism or tissue within the magnetic field may also be detected.

7. A method as claimed in claim 1, in which the biological organism is a mammal.

8. A method as claimed in claim 7, in which the mammal is either a human subject or a non-human subject.

9. A method as claimed In claim 1, in which the tissue is an electrically excitable tissue.

10. A method as claimed in claim 9, in which the tissue is a neural tissue.

11. A method as claimed in claim 9, in which the tissue is a tissue within the central nervous system of the subject.

12. A method as claimed in claim 11, in which the tissue is the brain of the subject.

13. A method as claimed in claim 10, in which the neural tissue is a peripheral nerve, or part of a peripheral nerve.

14. A method as claimed in claim 9 in which the tissue is a non-neural tissue.

15. A method as claimed in claim 14, in which the tissue is:
(a) the skin of the subject; or
(b) the heart or part of the heart of the subject.

16. A method as claimed in claim 14 in which the tissue is a muscle tissue.

17. A method as claimed in claim 16, in which the tissue is:
(a) skeletal muscle; or
(b) smooth muscle.

18. A method as claimed in claim 1, in which the biological organism is placed in either or both:
(a) a static magnetic field; and/or
(b) a time varying magnetic field

19. A method as claimed in claim 18 in which the time varying magnetic field, if used in the method, is present only during image acquisition by the magnetic resonance imaging scanner.

20. A method as claimed in claim 19, in which the magnetic resonance imaging scanner measures the electrically excitable tissue.

21. A method as claimed in claim 20, in which the magnetic resonance imaging sequence used in the performance of the method is:
(a) a spin-echo sequence; or
(b) a gradient echo sequence.

22. A method as claimed in claim 1, in which the electrical indicator is either or both:
(a) a direct electrical indicator of characteristics of function of the tissue; or
(b) an indirect electrical indicator of such function.

23. A method as claimed in claim 22, in which the method involves the use of a direct electrical indicator.

24. A method as claimed in claim 23, in which the indicator used comprises one or more of the following:
(a) the electroencephalogram;
(b) the electrocardiogram;
(c) the electromyogram; and/or
(d) measurements of skin conductance.

25. A method as claimed in claim 22, in which the method involves the use of an indirect electrical indicator.

26. A method as claimed in claim 1, in which the method permits the simultaneous or concomitant detection and recording over time in a magnetic resonance imaging acquisition sequence of the tissue or biological organism under observation, as well as changes over time in electrical observations on the tissue or biological organism.

27. A method as claimed in claim 2, in which the output of readings taken by the magnetic resonance imaging scanner is:
(a) displayed on a display means;
(b) recorded for subsequent use on a recording means; and/or
(c) printable on a printing means.

28. A method as claimed claim 1, in which the method comprises the simultaneous or concomitant capture of functional magnetic resonance imaging and electroencephalogram data from the biological tissue or organism under observation.

29. A method as claimed in claim 28, in which the method comprises the steps of:
(a) capturing functional magnetic resonance imaging information; and
(b) selectively sampling readings taken from an electroencephalogram taken in association with the magnetic resonance imaging procedure.

30. A method as claimed in claim 29, in which the method additionally comprises the use of means that are capable of distinguishing between:
(a) the electroencephalogram signal; and
(b) artifacts or signals which do not represent the electroencephalogram signal.

31. A method as claimed in claim 30, in which the means are capable of distinguishing:
(a) the electroencephalogram signal; and
(b) artifacts or signals which do not represent the electroencephalogram signal capture sample readings from the electroencephalogram signal during periods of time when artifacts are either wholly or substantially absent.

32. A method as claimed in claim 31, in which the method additionally comprises the use of either or both of the following:
(a) filtering means to filter unwanted data; and/or
(b) post-data capture processing means, in order to present data captured from observations taken on the biological tissue or organism.

33. A method as claimed in claim 32, in which to improve the accuracy of the results derived by using the method, the method additionally comprises the use of:
(a) a signal which is absent of the electroencephalogram signal, but which contains motion-related data; and/or
(b) artifact signal.

34. An apparatus suitable for performing the method claimed in claim 1.

35. An apparatus as claimed in claim 34, in which the apparatus comprises, or co-operates with means for detecting or monitoring the characteristics of at least one electrical indicator of the function of a tissue in a biological organism in the presence of a magnetic field over time.
